(19) United States
(12) Patent Application Publication
(10) Pub. No.: US 2010/0262007 A1
(43) Pub. Date: Oct. 14, 2010

(54) MEDICAL SCANNING APPARATUS AND METHOD

(75) Inventors: Andrew John Medlin, South Australia (AU); John Brydon, South Australia (AU); Matthew McCarthy, South Australia (AU)

Correspondence Address: Intellectual Property Dept.;Dewitt Ross & Stevens SC, 2 East Mifflin Street, Suite 600, Madison, WI, 53703-2865, US

(73) Assignee: SIGNOSTICS PTY LTD, Thebarton (AU)

(21) Appl. No.: 12/740,127

(22) PCT Filed: Oct. 31, 2008
(86) PCT No.: PCT/AU2008/001602

§ 371 Date: Apr. 28, 2010

(30) Foreign Application Priority Data
Nov. 2, 2007 (AU) 2007906066
Dec. 12, 2007 (AU) 2007906775

(51) Int. Cl. A61B 8/14

(52) U.S. Cl. 600/446

(57) ABSTRACT

A system and method of ultrasound scanning to achieve apparent real-time display of a sector ultrasound scan Including a single scanline ultrasound scanning apparatus with a sensor adapted to sense the position and/or orientation of the transducer and to output this as data and a screen to display a B-mode ultrasound scan image formed from a series of scanlines, each scanline being a dataset having a series of echo intensity values and a direction captured by reciprocating a transducer probe against a surface of a subject to be scanned such that a planar sector or series of planar sectors of the subject are swept by an ultrasound beam producing a series of scanlines and continuously processing and displaying said scanlines to produce an image that changes in real time In accordance with the features of the subject in the sector being scanned.

### TECHNICAL FIELD

This invention relates to the field of medical ultrasound scanning devices, In particular to low cost ultrasound devices which provide moving images of the scan field.

### BACKGROUND ART

The use of ultrasound scanning of patients for medical diagnostic purposes dates to the mid-20th century. An ultrasound transducer is used to project a beam of ultrasound energy into a patient. The same or another transducer detects the echoes returned from features within the body. These echoes, called a scanline, are then converted to a form suitable for recording or display.

When a series of scanlines, spaced angularly apart, are acquired rapidly and displayed on a display screen, the familiar B-mode sector scan is achieved.

This is a sector of a circle, wherein the brightness of each pixel of the display is proportional to the magnitude of the ultrasound echo received from the corresponding point in the body being imaged.

A method from the prior art for collecting the required series of scanlines which are spaced angularly apart was to provide a single transducer in a handpiece attached to a data processing and display unit by an articulated arm. The articulated arm included means at each joint for tracking the movement of the joint. Tracking the position of each joint allowed the position and orientation of the handpiece, and hence of the transducer, to be known at all times. An operator would place the handpiece against the body of a patient, and sweep the handpiece in an arc to obtain the required set of angularly spaced scanlines.

A further development of this method was to place a motor in the handpiece. This motor moved the transducer, relative to the handpiece. The transducer rotated about an axis parallel to the surface of the body to be scanned. Means were provided within the handpiece for accurately determining the positron of so the transducer relative to the handpiece as the transducer moved.

In use, an operator placed the handpiece against the body of a patient, and held the handpiece still. The rotating transducer was activated to cause the ultrasound beam emitted by the transducer to sweep out a sector of the body. All the scanlines obtained in a single sector sweep were displayed to form a static B-mode image.

Since the relative position of the transducer was now known, it was no longer necessary for the exact position and orientation of the handpiece to be known, and the articulated arm was no longer required.

A further method for collecting the required angularly separated scanlines came with the advent of transducer arrays consisting of a number of piezo-electric crystals where the transmitting pulse can be delayed in sequence to each crystal and thus effect an electronic means to steer the ultrasound beam. An operator uses the system in the same way as for the motor driven transducer. The steered beam sweeps out a sector to produce the static B-mode scan, without the need for a motor to move the transducer.

It is advantageous for a user to be able to see the area being scanned in “real-time”, that is, to see the display as a moving image of the area which is being scanned. This is commonly achieved by rapidly and continuously acquiring a series of static B-mode scans. These are then displayed sequentially, at approximately the rate at which they are acquired, that is, in real time.

The rate of display of the static B-mode scans must be at least sufficiently rapid such that the eye of the operator does not detect the transitions between scans. Since each scan needs to be assembled from its constituent scanlines, processed and displayed, the computer power required for this process is significant.

The motor driven transducer has the disadvantage that the motor and the associated moving parts decrease the reliability and increase maintenance requirements. The motor also adds cost, and more importantly, weight and bulk to the handpiece. The motor will also add significantly to the power consumption so of the ultrasound scanning system.

Nearly all modern medical ultrasound systems use an array of ultrasonic crystals in the transducer. The early designs used at least 64 crystals, with modern designs sometimes using up to a thousand crystals or more.

However, the cost of producing transducers with arrays of crystals is high. There is also a high cost in providing the control and processing circuitry, with a separate channel being required for each crystal. The transducers are usually manually manufactured, with the channels requiring excellent channel to channel matching and low cross-talk. The power consumption for electronic systems is also high, and is generally proportional to the number of channels to being simultaneously operational.

These problems make the use of such systems in hand held, battery powered applications infeasible.

### DISCLOSURE OF THE INVENTION

Compared to the prior art methods using motor driven transducers or array transducers, a simpler, lower cast way of acquiring a static B scan is to move a single crystal probe through a planar sector by hand. If this is performed in a reciprocating manner, it can produce an image that changes in real time, at a relatively low rate. To provide a smoothly changing image at these low refresh rates, the display is preferably not updated only at the end of each scan. It is advantageous to update the display as soon as each ultrasonic pulse echo returns to the sensor.

Therefore, In one form of the invention, although it may not be the only or the broadest form, the invention may be said to lie in an ultrasound scanning system including a transducer adapted to transmit ultrasound energy into a subject to be scanned, and to receive echoes returned from features of the subject;

a sensor adapted to sense the position and/or orientation of the transducer and to output this as data;

a processor adapted combine echo intensity data from the transducer with orientation data from the sensor to produce a scanline dataset;

a display and processing unit including digital data storage adapted to store an array of pixel elements, each pixel element having a value, that value determining the relative brightness of an associated pixel on a display screen, the display and processing unit being adapted to receive the scanline dataset and to process the scanline dataset to set values for each pixel element to cause the display of the scanline dataset on the display screen as part of a B-mode image;

wherein each scanline is transmitted to the display and processing unit at substantially the time it is produced and the scanline dataset is processed and displayed at substantially the time it is received by the display and processing unit.

However, to view a continuously updating display a means of removing old data from the display must be effected. One means is to allocate each pixel of the display a certain lifetime, after which it is deleted. Such deletion can either be instantaneous after a certain, fixed time or its display brightness can be reduced progressively.

is in preference scanline datasets are processed and displayed in groups, said groups being of a number substantially less than a number of scanline datasets which constitute a full B-mode image.

In preference the values for all pixel elements not set by the most recently processed group of scanline datasets are reduced by a uniform amount when that most recent group is processed for display.

In preference, in the alternative, each pixel element value is a function of any value in a most recently received scanline dataset where that value is associated with the pixel element and also of a plurality of past values of the pixel element; where the contribution of each said past value decreases with the time elapsed since that past value was the pixel element value.

In preference, the function follows a past time exponential law.

In preference, each pixel element value is determined by the sum of a first term proportional to any value in a most recently received scanline dataset associated with the pixel element and a second term proportional to an immediately preceding past value of the pixel element.

In a further form, the invention may be said to lie in a method of ultrasound scanning to achieve apparent real-time display of a B-mode ultrasound scan. Advantageously, this can be achieved in a low cost manner by employing a single scanline ultrasound scanning apparatus adapted to display a B-mode ultrasound scan image by displaying a series of scanlines, each scanline being a dataset having a series of echo intensity values and a direction.

In use, a user reciprocates a transducer of the scanning apparatus against a surface of a subject to be scanned such that a planar sector or series of planar sectors of the subject are swept by an ultrasound beam producing the required series of scanlines. By continuously processing and displaying the scanlines, an image is produced that changes in real time in accordance with the features of the subject in the sector being scanned. Such continuous processing may be done in batches or groups, where the batch or group size is much less than the size of a full sector scan.

In preference the instantaneous value of the brightness of each point of the displayed image is related to a brightness value determined by an most recently received scanline and to previous brightness values of that point, a previous value contributing less to the instantaneous brightness value as the time since that previous value was set becomes greater.

In preference, the instantaneous value of the brightness of a point is determined by a past time exponential law.

### BRIEF DESCRIPTION OF THE DRAWINGS

For a better understanding of this invention it will now be described with respect to the preferred embodiment which shall be described herein with the assistance of drawings wherein;

FIG. 1 is an illustration of a hand held ultrasound system embodying the Invention.

FIG. 2 is a block diagram of the circuitry of the embodiment of FIG. 1.

FIG. 3 is an illustration of a scanline pattern and pixel display buffer of the embodiment of FIG. 1.

### BEST MODE FOR CARRYING OUT THE INVENTION

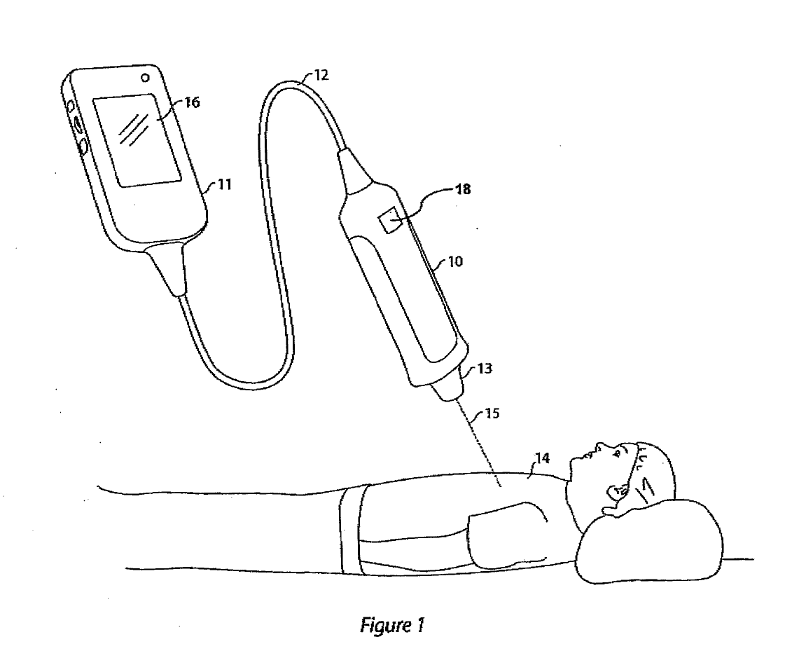

Referring now to FIG. 1, there is illustrated en ultrasound scanning system incorporating an embodiment of the invention. There is a hand held ultrasonic probe unit 10, a display and processing unit (DPU) 11 with a display screen 16 and a cable 12 connecting the probe unit to the DPU 11.

The probe unit 10 includes an ultrasonic transducer 13 adapted to transmit pulsed ultrasonic signals into a target body 14 and to receive returned echoes from the target body 14.

The transducer is adapted to transmit and receive in only a single direction at a fixed orientation to the probe unit, producing data for a single scanline 15. The system is a simple, low cost portable ultrasound scanning system. Additional transducers may be provided, at the expense of increased cost and complexity.

The probe unit further includes an orientation sensor 18 capable of sensing orientation or relative orientation about one or more axes of the probe unit. Thus, in general, the sensor is able to sense rotation about one or more of the axes of the probe unit.

The sensor may be implemented in any convenient form. In an embodiment the sensor consists of three orthogonally mounted gyroscopes. In further embodiments the sensor may consist of two gyroscopes, which would provide information about rotation about only two axes, or a single gyroscope providing information about rotation about only a single axis.

Since the distance between the mounting point of the sensor 18 and the tip of the transducer 13 is known, it would also be possible to implement the sensor with one, two or three accelerometers.

The orientation sensor may also incorporate position sensing, or may be only a position sensor, adapted to sense absolute position or movements in relative position of the probe unit. In further embodiments, the position and/or orientation sensor may be any combination of gyroscopes and accelerometers mounted in relative position to one another so as to give information about the linear and angular displacement of the probe unit. Full relative position data for the probe unit can be obtained with three orthogonally mounted accelerometers and three orthogonally mounted gyroscopes. This arrangement provides measurement of displacement in any direction and rotation about any axis. This allows for direction information for a scanline to be given for all six degrees of freedom.

in further embodiments, direction information for scanlines may be available for any number of possible degrees of freedom.

In another embodiment, the position and/or orientation sensing means is an electromagnetic spatial positioning system of the type requiring a fixed positioning transmitter separate from the probe unit, which transmits electromagnetic signals which are received by a receiver on the probe unit, the receiver providing information as to the position and orientation of the probe in the field of the transmitter.

The position and orientation means may be any suitable system or combination of systems which yields sufficient position information to form a useful image from the received scanlines. Optical positioning systems employing LED's and photodetectors may be used. This has the disadvantage of requiring line of sight access to the probe unit at all times.

Acoustic location systems may also be used combining a sound source on the probe with acoustic sensors at known points.

Visual tracking systems using a camera to observe the movement of the probe and translate this into tracking data may also be used. This has the disadvantage of requiring line of sight access to the probe unit at all times.

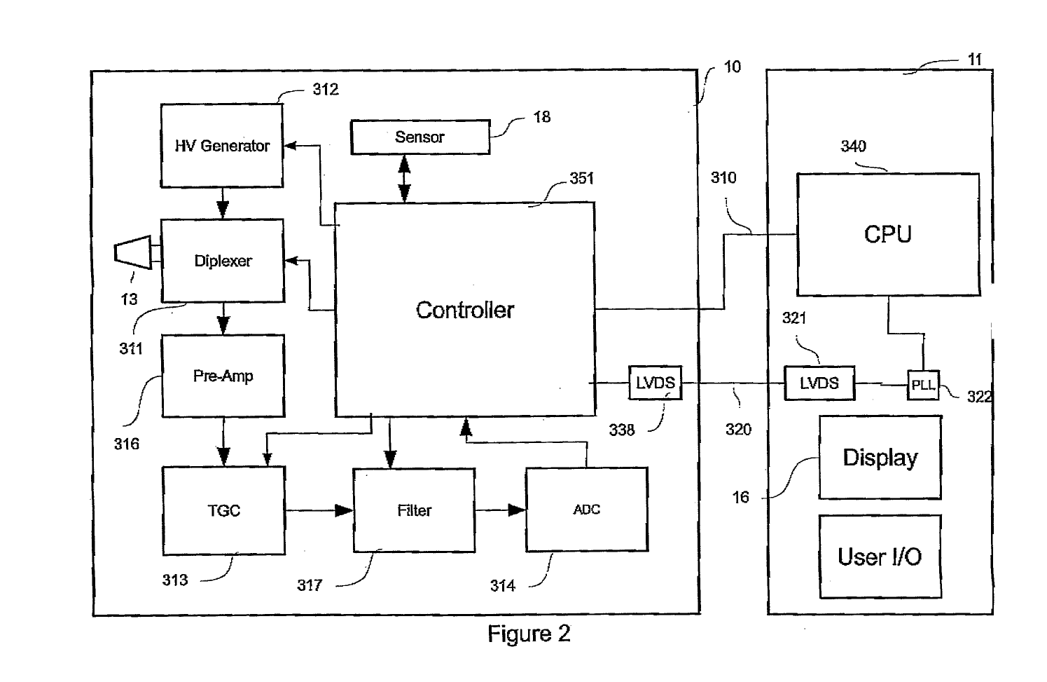

A block diagram of the ultrasonic scan system is shown in FIG. 2. There is a probe unit 10 and a Display and Processing Unit (DPU) 11. The probe unit includes a controller 351 which controls all of the functions of the probe.

The DPU includes a main CPU 340.

The probe unit 10 communicates with the DPU 11 via a low speed message channel 310 and a high speed data channel 320. The message channel is a low power, always on connection.

The data channel is a higher speed and hence higher power consumption bus which is on only when required to transmit data from the probe unit to the DPU. The probe unit includes a transducer 13 which acts to transmit and receive ultrasonic signals. A diplexer 311 is used to switch the transducer between transmit and receive circuitry.

On the transmit side the diplexer is connected to high voltage generator 312, which is controlled by controller 351 to provide a pulsed voltage to the transducer 13. The transducer produces an interrogatory ultrasonic pulse in response to each electrical pulse.

This interrogatory pulse travels into the body and is reflected from the features of the body to be imaged as an ultrasonic response signal. This response signal is received by the transducer and converted into an electrical received signal.

The depth from which the echo is received can be determined by the time delay between transmission and reception, with echoes from deeper features being received after a longer delay. Since the ultrasound signal attenuates in tissue, the signal from deeper features will be relatively weaker than that from shallower features.

The diplexer 311 connects the electrical receive signal to time gain compensation circuit (TGC) 313 via a pre-amp 316. The TGC applies amplification to the received signal. The characteristics of the amplification are selected to compensate for the depth attenuation, giving a compensated receive signal where the intensity is proportional to the reflectiveness of the feature which caused the echo. In general, the amplification characteristics may take any shape.

This compensated signal is passed to an analogue to digital converter (ADC) 314, via an anti-aliasing filter 317. The output of the ADC is a digital data stream representing the intensity of the received echoes over time for a single ultrasonic pulse.

There is an orientation sensor 18 which is adapted to provide information about angular rotation of the probe unit.

In use, a user applies the probe unit 10 to a body to be imaged 14. A scan is initiated by the user, by means of a control either on the probe unit or on the DPU. The activation of the control is detected by the controller 351 and communicated to the DPU 11 via the message channel 310.

The DPU responds with a message which includes any parameters which have been selected for the scan. The controller 351 controls the high voltage driver to produce the required pulse sequence to be applied via the diplexer to the transducer in order to perform a scan according to the parameters set by the user, or set as defaults in the DPU.

The user rotates the probe as required to sweep the ultrasound beam over the desired area, keeping linear displacement to a minimum.

In embodiments where rotation about all axes is not sensed, the user will also keep rotation about unsensed axes, that is, axes about which rotation is not detected by the sensor of the embodiment, to a minimum.

At the same time, data is received from the orientation sensor 18. This is the rotation about the sensed axes of the probe unit. It may be the angular change in the position of the probe unit since the immediately previous transducer pulse, or the orientation of the probe unit in some defined frame of reference. One such frame of reference may be defined by nominating one transducer, pulse, normally the first of a scan sequence, as the zero of orientation.

The sensor data and the response signal are passed to the controller 351 where they are combined to give a scanline dataset. A scanline dataset comprises a sequential series of intensity values of the response signal combined with orientation information.

The scanline dataset is generated in the controller 351. The scanline dataset is then passed to a protocol converter to be converted to a protocol suitable for transmission via the data channel. Any suitable protocol may be used. In this embodiment the protocol chosen for use on the data channel is 8b10b, which is well known in the art.

The 8b10b data is passed to an LVDS transmitter 338 and is transmitted via the data channel 320 to the DPU 11.

The LVDS data channel is received by the DPU via LVDS receiver 321 and phase locked loop 322, The 8b10b data is passed to the DPU processor 340.

Protocol conversion is performed by processor 340 to recover the original scanline dataset.

An application is now run by the DPU processor 340 to process the scanline dataset for display on the display 16 of the DPU 11.

The high voltage generator 312 continues to provide the pulsed voltage to the transducer under control of the microcontroller and each pulse results in a scanline.

In order to produce a real time display, the user continues to scan the probe unit and hence the scanline, through a planar sector in a reciprocating manner. The transducer is continuously pulsed, providing a stream of scanline datasets which are displayed on the display 16 to form a real time moving image.

The pulsing of the transducer, and hence the rate at which scanlines are produced, may be determined based on time, or on angle through which the transducer has been moved, or by a combination of these factors.

The rate at which a full sector scan can be produced is limited by the speed at which the user reciprocates the probe unit. This will be significantly slower than the rate at which prior art devices employing motor driven transducers or swept array transducers provide sector scans.

To provide a smoothly changing image at these low refresh rates, the display is updated as each scanline dataset is received and processed by the DPU 11, rather than replacing each sector scan in its entirety with the next sector scan as in the prior art. Waiting for each sector scan to be available in it entirety before displaying any of that scan would result in an unacceptably jumpy display, in the case where each sector scan may take some seconds to acquire.

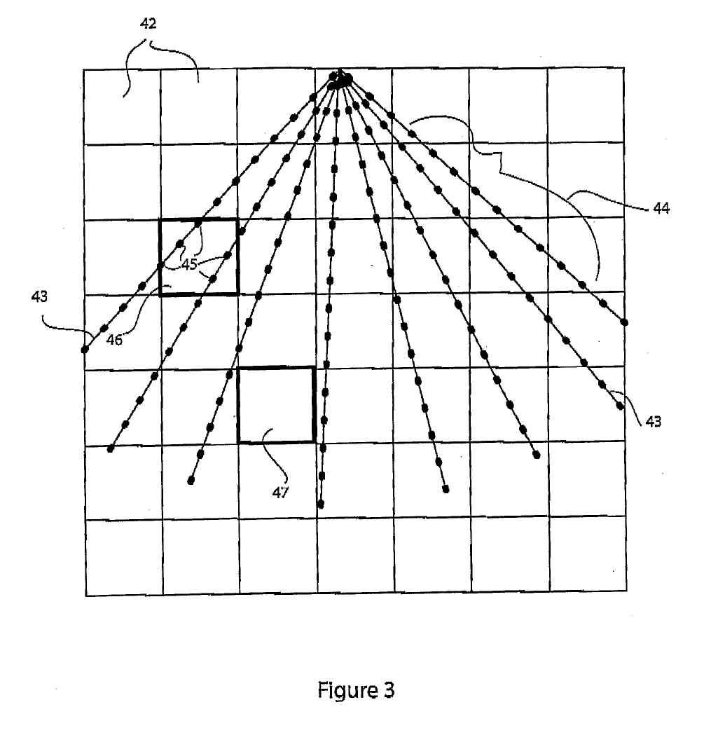

The display 16 is associated with a pixel buffer 41, shown diagrammatically in FIG. 3. The pixel buffer is an array of pixel elements 42. Each pixel element has a scalar data value which used to set the brightness of a corresponding physical pixel an the display 16.

In order to display a B-mode sector scan image, scanlines 43 are overlaid onto so the pixel buffer. Each scanline is a series of acoustic reflection intensity values 44, at a direction defined by the orientation sensor 18.

It can be seen that multiple scanline data points 45 may be overlaid onto a single pixel element 46. The scanline data is processed such that each pixel element has one and only one value p_{xy} contributed to by the scanline data.

Any suitable method may be chosen to make this association. Possible methods include averaging the values of all of the data points which occur in a pixel element, choosing the modal value of all of the data points which occur in a pixel, or choosing an arbitrarily defined value, for example the one nearest the centre of the pixel element. Many other more complex methods for defining the value of the pixel element are possible.

There are also pixel elements 47 which do not directly align with any scanline. In order to avoid the image being split at this point by an apparent black line which would not correspond to any real feature, values for these blank elements may be interpolated from surrounding pixel elements. Any suitable method of interpolation may be used to assign values to these pixel elements.

As new scanline data is received, the new data is processed and a new value for all affected pixel elements pnew_{xy}(t) is calculated. However, unlike in a prior art real time B-mode scan, a new full B-mode sector scan is not available, only a single scanline. Thus all of the pixel elements cannot be updated simply based on the newly received scanline, since this would blank the screen except in the vicinity of the newly received scanline.

The new scanline data may be processed as each single scanline dataset is available, or may be processed in batches, each batch being sufficiently small that the display is not made noticeably discontinuous.

In order to achieve a smooth, coherent image while continuously updating the display a means of controlled, time related updating of the data in the pixel buffer is implemented.

Each pixel element value may be allocated a certain lifetime, after which it is reset to zero. In an embodiment, the zeroing is instantaneous after a certain, fixed time.

In a preferred embodiment, the value is reduced progressively, with all pixel values being reduced as each batch of scanline data is processed.

In use, a user first sets the desired depth of a scan sector, or accepts a default depth setting. The user then begins a scan by holding the transducer at the approximate centre point of the desired scan sector, and initiating a scan. The user then rotates the transducer so as to sweep out a scan sector, approximately in a single plane.

The DPU begins to receive scanline data. Display of the scanlines does not begin until a sufficient batch of scanlines has been received. The size of the batch may be defined by a selected number of scanlines, by a selected angle being swept by the transducer, or by the expiry of a selected time, say 100 milliseconds. More complex combinations of these parameters may be used to define batch size, which need not be constant for the duration of a scan.

When a batch of scanlines has been received, the scanlines are displayed. The first scanline received is taken as a datum line, and is displayed directly down the screen, with the following lines distributed at the angles determined by the angle data within each scanline dataset.

When a subsequent batch of scanlines is received, all non-zero pixel values within the pixel buffer are reduced by a selected percentage, say five percent. The new batch of scanlines is shown at the received intensity, without reduction.

This gives a “radar sweep” effect, whereby the brightness of the most recently displayed pixels serves to indicate approximately from where in the sector the scanline is being received.

The process is repeated for each batch of scanlines received. The last scanline or the last few scanlines from the immediately preceding batch may be included in each batch, in order that the interpolation of values for pixels which do not have a directly corresponding data point in a scanline dataset, as shown in FIG. 3, can be undertaken.

When the user has rotated the probe unit as far as desired, or as far as practical, the user will then begin to sweep in the opposite direction, sweeping back over areas already scanned and displayed on the screen.

These scanlines are displayed in the same way, with the pixel values calculated from the new batch replacing those from previous batches which are within the same angular area measured from the datum line.

The user continues to sweep the transducer over the area to be imaged, changing direction at the extreme angle of each sweep, and batches of scanlines are continuously processed and displayed.

Since the fading is applied as a percentage of the current value, the value of brightness for pixels which are not overwritten decays exponentially.

In a further embodiment the value of each pixel element is reduced progressively, following a past time exponential law:

$$y ( t - T ) = y ( T ) \cdot ( 1 - {e}^{\frac{- ( t - T )}{τ}} )$$

for t>T, Where y(T) is the original pixel buffer value corresponding to the amplitude of the echo received at time, T.

From time to time the current value of a pixel element will be augmented with a is newly allocated value. This will be when a scanline with a direction causing it to fall within the pixel is received and processed. The new component of value will then start to decay in value following a past time exponential law from its time of updating.

Typically, time constant, τ, is of the order of one pass of the sensor, say approximately 4 seconds.

Since the pixel element value is proportional to the brightness of the corresponding pixel on the display, it can be seen that a feature in the image that is not refreshed fades away on the display, while one that is repeatedly scanned remains visible.

This allows the easy visualisation of adjacent planes within the body by reciprocating the probe while progressively altering the angle of the plane which it sweeps.

The DPU 11 implements the past time exponential decay in pixel element brightness value by filtering each pixel element 42 of the pixel buffer in place with a recursive digital filter of the form:

p_{xy}((n+1)T)=pnew_{xy}(nT)+α·p_{xy}(nT)

where, pnew_{xy}(t) is any new input to the pixel element, and p_{xy}(t) the value of the pixel element at time t.

Thus, at each time interval, nT, the updated value of each pixel element is equal to the value of any new input plus a constant times the previous value. The speed at which the past signals fade is set by coefficient, α.

It is desirable to have the facility to save a B-mode image produced by the device. This may be for later study, for enhancement for further study, for record keeping or for other purposes. It is possible to simply save the state of the pixel buffer at the completion of the scan, but this would have the undesirable effect that the fading of some pixels would be recorded.

In order to record the best possible image it is desirable to save the pixel buffer data covering a full sector as swept by the user. Since a user is likely to end a scan part way through a sector sweep, a means is provided to make available a full sector sweep, which includes the latest available scanlines.

As scanline data is received, it is stored in a Current Sweep Buffer. When the user reverses the sweep direction, this data is transferred to a buffer called Previous Sweep Buffer. When the user again reverses direction, this process is repeated. Accordingly, at any time there will be one buffer containing a full sector sweep and a second buffer containing the most recent scanline datasets which do not constitute a full sector sweep.

In order to assemble the final pixel buffer data for recording, the two buffers are combined, with the data in the Current Sweep Buffer having precedence. That is, where there is a value for a pixel element available from the Current Sweep Buffer that will be used in the recorded data where there is no such value, the value from the Previous Sweep Buffer will be used. In neither case will any fading or reduction in intensity value be applied.

The final combination of the data from the two buffers will become the final recorded sector sweep image.

In practice, the transfer of the values between the Current Sweep Buffer and the Previous Sweep Buffer may take place by renaming the buffers, by changing pointers or by any other convenient means, not necessarily involving rewriting all of the computer memory occupied by the buffer data.

The decision that the user has indeed reversed the direction of sweep of the transducer, and that the buffers should be changed includes a threshold check of the amount of angle reversal to ensure that hand shake or momentary reversal of direction does not cause the previous buffer data to be discarded prematurely.

Although the invention has been herein shown and described in what is conceived to be the most practical and preferred embodiment, it is recognised that departures can be made within the scope of the invention, which is not to be limited to the details described herein but is to be accorded the full scope of the appended claims so as to embrace any and all equivalent devices and apparatus.

1-19. (canceled)

20. An ultrasound scanning system including:
a. a transducer configured to:
(1) transmit ultrasound energy into a subject to be scanned,
(2) receive resulting echoes returned from features of the subject, and
(3) generate echo data therefrom;
b. a sensor configured to:
(1) sense the position and/or orientation of the transducer, and
(2) generate position and/or orientation data therefrom;
c. a processor configured to generate a scanline dataset combining:
(1) the echo data, and
(2) the position and/or orientation data;
d. a display and processing unit:
(1) including digital data storage configured to store an array of pixel elements, each pixel element having a value determining the relative brightness of an associated pixel on a display screen,
(2) being configured to:
(a) receive the scanline dataset at least substantially simultaneously with the generation of the scanline dataset in the processor;
(b) process the scanline dataset to define values for pixel elements corresponding thereto, and
(c) generate a display of the scanline dataset on the display screen:
(1) as part of a B-mode image, and
(2) at least substantially simultaneously with the reception of the scanline dataset in the display and processing unit.

21. The system of claim 20 wherein each scanline dataset is processed and displayed within a group of one or more scanline datasets, the group containing a number of scanline datasets which is substantially less than a number of scanline datasets which constitute a complete B-mode image.

22. The system of claim 20 wherein the values for all pixel elements whose values were not set during the processing of the scanline dataset are reduced prior to generating the display.

23. The system of claim 20 wherein the value for each pixel element whose value was not set during the processing of the scanline dataset is reduced by a fixed percentage of the pixel element's current value prior to generating the display.

24. The system of claim 20 wherein the display and processing unit is further configured to generate, store and display a static B-mode image defined by:
a. the scanline dataset, and
b. previously-generated scanline datasets,
wherein the brightnesses of the pixels of the B-mode image are proportional to data values within the scanline datasets.

25. The method of claim 24 wherein the display and processing unit is configured to:
a. evaluate the position and/or orientation data to determine the occurrence of a reversal of direction of motion of the transducer probe, and
b. determine which of the previously received scanline datasets shall be included in the static B-mode image with reference to this occurrence.

26. The system of claim 20 wherein the value of each pixel element is a function of:
a. the most recently received scanline dataset corresponding thereto, and
b. at least one prior scanline dataset corresponding thereto, wherein the contribution of each prior scanline dataset decreases with its age in relation to the most recently received scanline dataset.

27. The system of claim 26 wherein the contribution of each prior scanline dataset is set to zero once the prior scanline dataset attains a predefined age in relation to the most recently received scanline dataset.

28. The system of claim 26 wherein the contribution of each prior scanline dataset decreases in accordance with an exponential relation.

29. The system of claim 20 wherein the value of each pixel element is at least partially defined by a sum of:
a. a corresponding value in the most recently received scanline dataset, and
b. a corresponding value in a prior scanline dataset.

30. The system of claim 29 wherein each pixel element value is determined by applying a recursive digital filter of the form:
p_{xy}((n+1)T)=pnew_{xy}(nT)+αp_{xy}(nT)
wherein:
a. pnew_{xy}(t) is a corresponding value in the most recently received scanline dataset at time t;
b. p_{xy}(t) is a corresponding value in a prior scanline dataset at time t; and
c. α is a constant.

31. The system of claim 20 wherein each pixel element value is set to zero a predetermined time after its initial display.

32. An ultrasound scanning system including:
a. a transducer configured to:
(1) transmit ultrasound energy into a subject to be scanned,
(2) receive resulting echoes returned from features of the subject, and
(3) generate echo data therefrom;
b. a sensor configured to generate position and/or orientation data corresponding to the position and/or orientation of the transducer;
c. a display and processing unit configured to generate a B-mode image to be displayed on a display screen, the B-mode image being formed of pixel brightnesses defined by:
(1) one or more current scanlines combining:
(a) the most recently generated echo data, and
(b) the most recently generated position and/or orientation data,
(2) one or more prior scanlines combining:
(a) previously-generated echo data, and
(b) previously-generated position and/or orientation data,
wherein the pixel brightnesses defined by prior scanlines decrease with their age in relation to the current scanlines.

33. A method of ultrasound scanning to achieve apparent real-time display of a sector ultrasound scan including the steps of:
a. providing a single scanline ultrasound scanning device configured to display a B-mode ultrasound scan image formed from a series of scanlines, each scanline being a dataset having a series of echo intensity values and a direction;
b. reciprocating a transducer probe of the scanning device against a surface of a subject to be scanned such that one or more planar sectors of the subject are swept by an ultrasound beam to produce a series of scanlines;
c. continuously processing and displaying the scanlines to produce an image that changes in real time in accordance with the features of the subject in the sector being scanned.

34. The method of claim 33 wherein:
a. each displayed scanline has a brightness value, and
b. the brightness value is reduced by an amount proportional to the length of time for which the scanline has been displayed.

35. The method of claim 33 wherein:
a. the displayed image is defined by pixels, and
b. each pixel of the displayed image has a current brightness value defined by:
(2) a most recently received scanline, and
(2) prior brightness values of the pixel,
wherein prior brightness values contribute less to the current brightness value as the prior brightness values grow older.

36. The method of claim 33 wherein each displayed scanline is composed of pixels, each pixel having a brightness value which decreases over time in accordance with an exponential relation until a new scanline corresponding to the pixel is produced.

37. The method of claim 33 wherein each displayed scanline is composed of pixels, each pixel having a brightness value determined by:
$$y ( t - T ) = y ( T ) \cdot ( 1 - {e}^{\frac{- ( t - T )}{τ}} )$$
for t>T, where y(T) is the brightness value of the pixel of the image at time T.

38. The method of claim 33 further including the steps of storing and displaying a static B-mode ultrasound scan image having pixels with brightnesses proportional to data values within the scanline datasets.

39. The method of claim 38 further including the steps of:
a. using the direction information within successively received scanline datasets to determine a direction of motion of the transducer probe,
b. determining when the direction of motion reverses, and
c. using the time of such reversal in relation to the time when each scanline dataset is received to determine which scanline datasets contribute to the static B-mode image.
